(19) United States
(12) Patent Application Publication
(10) Pub. No.: US 2026/0284655 A1
(43) Pub. Date: Sep. 24, 2026

(54) CHANNEL ELEMENT, PACKAGE, DRUG DELIVERY DEVICE, BIOMOLECULE TESTING APPARATUS, AND CHANNEL FORMING METHOD

(71) Applicant: OKINAWA INSTITUTE OF SCIENCE AND TECHNOLOGY SCHOOL CORPORATION, Kunigami-gun, Okinawa (JP)

(72) Inventors: Stoffel Dominique JANSSENS, Kunigami-gun, Okinawa (JP); David VAZQUEZ-CORTES, Kunigami-gun, Okinawa (JP); Burhannudin SUTISNA, Kunigami-gun, Okinawa (JP); Eliot FRIED, Kunigami-gun, Okinawa (JP)

(73) Assignee: OKINAWA INSTITUTE OF SCIENCE AND TECHNOLOGY SCHOOL CORPORATION, Kunigami-gun, Okinawa (JP)

(21) Appl. No.: 19/477,577

(22) PCT Filed: Apr. 26, 2024
(86) PCT No.: PCT/JP2024/016586

§ 371 Date: Oct. 22, 2025

(30) Foreign Application Priority Data
Apr. 27, 2023 (JP) 2023-073856

(51) Int. Cl. B01L 3/00; A61M 31/00; H05K 1/02

(52) U.S. Cl. CPC B01L 3/502715; A61M 31/002; B01L 3/502707; B01L 2200/12; B01L 2300/0645; B01L 2300/12; H05K 1/0284

(57) ABSTRACT

A channel element according to the present disclosure, comprises a substrate, a film formed on the substrate, and a transformed portion of a part of the film, the transformed portion extending in line shape along the substrate. First channels are formed along the transformed portion between the substrate and the film delaminated on both sides of the transformed portion.

### CROSS-REFERENCE TO RELATED APPLICATION

The present application claims priority from Japanese Application No.2023-073856, filed on Apr. 27, 2023, the content of which is incorporated herein by reference.

### TECHNICAL FIELD

The present disclosure relates to a channel element, a package, a drug delivery device, a biomolecule testing apparatus, and a channel forming method.

### BACKGROUND

Methods for manufacturing channel elements (also called “channel devices” or “channel structures”) with fine channels such as microchannel elements and nanochannel elements have been proposed (for example, see Non-Patent Literatures (NPL) 1-3 and Patent Literature (PTL) 1. The channel elements are expected to be applied in a wide range of fields, such as the analysis of biomolecules and the synthesis of minute amounts of substances. In particular, in the field of nanofluidics, focused ion beam (FIB), electron beam lithography (EBL), ultraviolet (UV), and deep ultraviolet lithography are often used to form channel structures with dimensions from hundreds of to tens of nanometers. Furthermore, direct laser writing of nanochannels using lasers, such as femtosecond lasers, is expected as a method to form extremely fine channels (For example, see Non-Patent Literatures (NPL) 2 and 3).

### CITATION LIST

### Patent Literature

PTL 1: Japanese Patent Application Laid-open No. 2014-240065

### Non-Patent Literature

NPL 1: Chen, L.; Yang, C.; Xiao, Y.; Yan, X.; Hu, L.; Eggersdorfer, M.; Chen, D.;

Weitz, D.; Ye, F. Millifluidics, Microfluidics, and Nanofluidics: Manipulating Fluids at Varying Length Scales. Mater. Today Nano 2021, 16, 100136.
NPL 2: Yamamoto, K.; Ota, N.; Tanaka, Y. Nanofluidic Devices and Applications for Biological Analyses. Anal. Chem. 2021, 93, 332-349.
NPL 3: Sima, F.; Sugioka, K. Ultrafast Laser Manufacturing of Nanofluidic Systems. Nanophotonics 2021, 10, 2389-2406.

### SUMMARY

### Technical Problem

However, there are some points to be improved in the conventional manufacturing methods for channel elements. For example, in conventional methods, a pattern of open-topped channels is first formed on a substrate. A processing method such as electron beam lithography or focused ion beam milling is used to form this pattern. Optical lithography and etching are also used to form wider channels. After forming the open-topped channels in the substrate in this manner, a step of sealing the formed channels is performed. As a method for sealing the channels, for example, a method such as anodic bonding is employed for bonding a glass plate onto the substrate (for example, see Non-Patent Literature (NPL) 1 and Patent Literature (PTL) 1). As described above, the fabrication of channel elements is complex and requires many steps, and thus is associated with high costs.

Accordingly, an object of the present disclosure, which focuses on the above-mentioned points, is to provide a method for manufacturing a channel element with fewer steps, a channel element manufactured by the method, and devices incorporating the channel element such as, for example, a package, a drug delivery device, and a biomolecular testing apparatus.

### Solution to Problem

The gist of the present disclosure is as follows:
[1] A channel element, comprising:
   a substrate;
   a film formed on the substrate; and
   a transformed portion of a part of the film, the transformed portion extending in line shape along the substrate, wherein
   first channels are formed along the transformed portion between the substrate and the film that is delaminated on both sides of the transformed portion.
[2] The channel element according to [1], wherein
   the transformed portion has a mass density lower than that of the film.
[3] The channel element according to [1] or [2], wherein
   the film includes a layer adjacent to the substrate having a higher absorption rate of light of a predetermined wavelength than the other part of the film.
[4] The channel element according to any one of [1] to [3], wherein
   the substrate is a glass substrate.
[5] The channel element according to any one of [1] to [4], wherein
   the film is a diamond film.
[6] The channel element according to [5], wherein
   the transformed portion includes non-diamond carbon.
[7] The channel element according to any one of [1] to [6], wherein
   the film has a thickness in a range of 145 nm to 1000 nm.
[8] The channel element according to any one of [1] to [7], wherein
   the transformed portion has a width in a range of 0.5 μm to 5 μm.
[9] The channel element according to any one of [1] to [8], wherein
   a dimension of the first channel in a direction perpendicular to the substrate is in a range of 5 nm to 200 nm.
[10] The channel element according to any one of [1] to [9], wherein the
   width of the first channel in a direction along the substrate is in a range of 1 μm to 4μm.
[11] The channel element according to any one of [1] to [10], further comprising
   two second channels having a larger dimension than the first channels each connected to a reservoir capable of storing a fluid, wherein
   the first channels connect between the two second channels.
[12] A package comprising the channel element according to any one of [1] to [11], wherein the first channels connect between an internal space of the package in which an electronic component can be arranged and a space outside the package.
[13] A drug delivery device comprising the channel element according to any one of [1] to [11], wherein the first channel is used as a channel for drug delivery.
[14] A biomolecule testing apparatus comprising:
   the channel element according to any one of [1] to [11]; and
   a plurality of electrodes provided at a distance from each other along the first channels of the channel element.
[15] A channel forming method including:
   forming a film on a substrate; and
   irradiating the film with a laser light to form a transformed portion in line shape in a part of the film, wherein the transformed portion causes delamination of the substrate and the film on both sides of the transformed portion to form first channels along the transformed portion between the substrate and the film.
[16] The channel forming method according to [15], wherein
   the portion of the film irradiated with the laser light expands to form the transformed portion and causes delamination of the substrate and the film on both sides of the transformed portion.
[17] The channel forming method according to [15] or [16], wherein
   the film includes a layer adjacent to the substrate having a higher absorption rate of the laser light than the other portions of the film.
[18] The channel forming method according to any one of [15] to [17], wherein
   the substrate is a glass substrate.
[19] The channel forming method according to any one of [15] to [18], wherein
   the film is a diamond film.
[20] The channel forming method according to any one of [15] to [19], wherein
   the laser light is applied to the film as a convergent beam that converges on an interface between the substrate and the film.
[21] The channel forming method according to any one of [15] to [20], wherein
   the laser light is a pulsed laser light having a pulse width in a range of 10⁻¹⁵ second to 10⁻¹² second.
[22] The channel forming method according to any one of [15] to [21], wherein
   energy of the laser light is selected from a range of energies in which the part of the film is transformed but the film is not removed by the laser light.
[23] The channel forming method according to any one of [15] to [22], further including
   filling the first channels with a liquid that corrodes the substrate to enlarge the first channels on both sides of the transformed portion to form a single third channel.

### Advantageous Effect

According to the present disclosure, the channel element can be fabricated in a simpler manner than in conventional technologies.

### BRIEF DESCRIPTION OF DRAWINGS

In the accompanying drawings:

FIG. 1 is a plan view of a channel element according to an embodiment.

FIG. 2 is a cross-sectional view of the channel element of FIG. 1 taken along the line A-A.

FIG. 3 illustrates a method for manufacturing the channel element.

FIG. 4 is a flow chart showing a method for manufacturing a nanofluidic device as a channel element according to an embodiment.

FIG. 5A is a plan view of a glass substrate used for fabricating a nanofluidic device.

FIG. 5B is a partial cross-sectional view of the glass substrate of FIG. 5A taken along the line B-B.

FIG. 6A is a plan view of a glass substrate after applying nanodiamond seed crystals.

FIG. 6B is a partial cross-sectional view of the glass substrate of FIG. 6A taken along the line C-C.

FIG. 7A is a plan view of an in-process nanofluidic device with a grown poly-crystalline diamond film.

FIG. 7B is a partial cross-sectional view of the in-process nanofluidic device of FIG. 7A taken along the line D-D.

FIG. 8A is a plan view of an in-process nanofluidic device after patterning.

FIG. 8B is a partial cross-sectional view of the in-process nanofluidic device of FIG. 8A taken along the line E-E.

FIG. 9A is a plan view of an in-process nanofluidic device with a further grown poly-crystalline diamond film.

FIG. 9B is a partial cross-sectional view of the in-process nanofluidic device of FIG. 9A taken along the line F-F.

FIG. 10A is a plan view of an in-process nanofluidic device after microchannel formation.

FIG. 10B is a partial cross-sectional view of the in-process nanofluidic device of FIG. 10A taken along the line G-G.

FIG. 11A is a plan view of a fabricated nanofluidic device.

FIG. 11B is a partial cross-sectional view of the nanofluidic device of FIG. 11A taken along the line H-H.

FIG. 12A is a plan view of the nanofluidic device after filling the channels with liquid.

FIG. 12B is a partial cross-sectional view of the nanofluidic device of FIG. 12A taken along the line I-I.

FIG. 13 is an electron microscope image of the end face of the cleaved nanofluidic device.

FIG. 14 is height profiles of laser-written structures versus laser power.

FIG. 15A is a plan view of a package using a channel element.

FIG. 15B is a cross-sectional view of the package of FIG. 15A.

FIG. 16A is a plan view of a drag delivery device using a channel element.

FIG. 16B is a cross-sectional view of the drug delivery of FIG. 16A.

FIG. 17 is a plan view of a biomolecule testing apparatus using a channel element.

FIG. 18A is a cross-sectional view of a channel element containing nanochannels.

FIG. 18B is a channel element comprising microchannels fabricated from the channel element of FIG. 18A.

### DETAILED DESCRIPTION

Hereinafter, embodiments of the present disclosure will be described with reference to the drawings.

### Channel Element

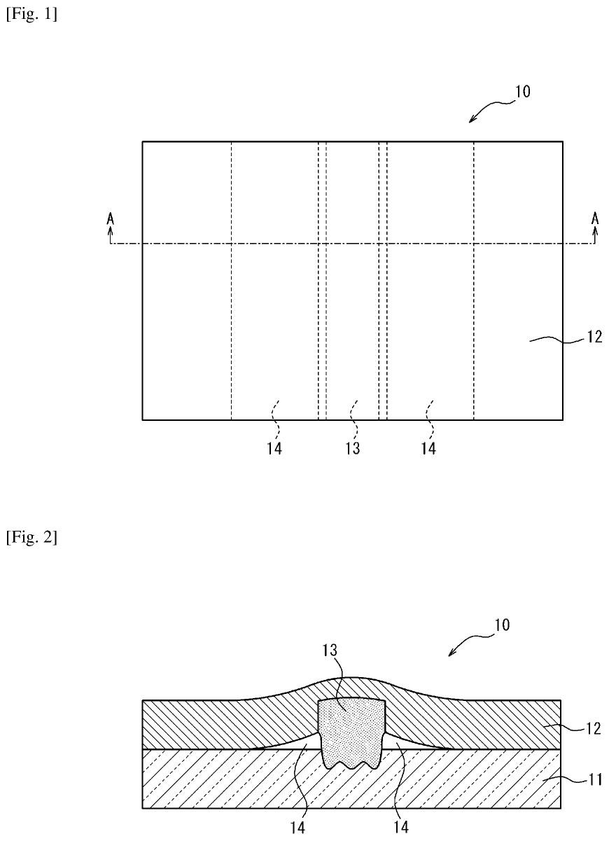

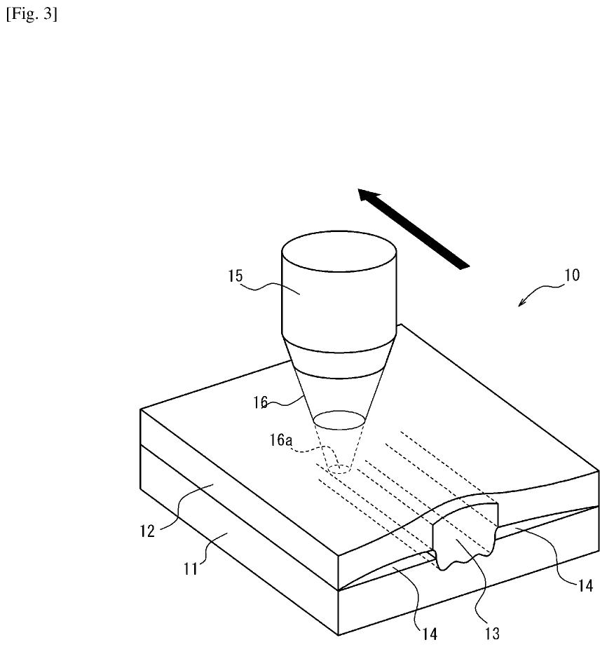

As shown in FIGS. 1 and 2, a channel element 10 according to an embodiment of the present disclosure includes a substrate 11, a film 12 formed on the substrate 11, and a transformed portion 13 of a part of the film 12. The transformed portion 13 extend in line shape along the substrate 11. First channels 14 are formed along the transformed portion 13 between the substrate 11 and the film 12 that are delaminated on both sides of the transformed portion 13.

The substrate 11 is a flat plate-like member. The substrate 11 can be a member transparent to visible light, such as glass. Substrate 11 is not limited to a transparent member.

The film 12 is formed on the substrate 11 by using any suitable fabrication method. Suitable methods of forming the film 12 include vapor growth methods and liquid growth methods. The vapor growth methods include, for example, chemical vapor deposition (CVD), evaporation methods, and sputtering. Liquid growth methods include, for example, plating, sol-gel methods, and spin coating.

The film 12 has an upper layer having a low absorption rate of light at a wavelength of a laser light 16, which will be discussed later, and a lower layer having an absorption rate higher than the upper layer. Here, the upper layer is a layer on the opposite side of the substrate 11, and the lower layer is a layer on the substrate 11 side. For example, the absorption rate of light at the wavelength of the laser light 16 of the film 12 may gradually change in the thickness direction so that it is highest on the substrate 11 side. For example, the film 12 may be composed of a material whose optical properties can be adjusted by controlling defect density. Moreover, the film 12 can be formed of a plurality of layers having different absorption rates of the light at the wavelength of the laser light 16. Any combination of materials that behave dif-ferently to laser lights of specific wavelengths and/or intensities can be used for making the nanochannels of the present disclosure. The film 12 preferably has high chemical and thermal robustness and biocompatibility. Compared to the substrate 11, the film 12 may be made of a material that is easily transformed by the laser light 16.

A material transparent to visible light can be used for the film 12. By forming the substrate 11 and the film 12 with materials transparent to visible light, it is possible to observe the inside of the channel 14 formed in the channel element 10 from the outside of the channel element 10. The film 12 may be, for example, a diamond film.

The transformed portion 13 includes the material of the film 12 transformed by irradiation with the laser light 16 in the fabrication process of the channel element 10. The transformed portion 13 may further include the material of the substrate 11. In FIG. 2, the transformed portion 13 is located only on the substrate 11 side of the film 12, but the transformed portion 13 may exist over the entire thickness of the film 12. Unlike FIG. 2, the boundary between the transformed portion 13 and the film 12 is not clear in many cases. Also, the degree of transformation of the film 12 may vary depending on the position in the transformed portion 13. That is, the transformed portion 13 need not be uniformly transformed. Maximum transformation of the transformed portion 13 from the material of the film 12 may occur on the side of the transformed portion 13 that is in contact with the substrate 11.

A typical width of the transformed portion 13 is in the range of 0.5 μm to 5 μm. This width of the transformed portion 13 is on the order of the size of the focal spot 16a of the laser light 16. In practice, it may be difficult to reduce the size of the focal spot 16a to less than 0.5 μm. The use of laser light 16 with a larger focal spot size results in an unnecessarily large transformed portion 13 and in larger energy consumption. The width of the transformed portion 13 can be enlarged by increasing the laser beam intensity. However, exceeding a certain laser beam intensity will result in complete film 12 removal. The width of the transformed portion 13 is not limited to 0.5 μm to 5 μm as described above. If the focal spot 16a of the laser light 16 can be made smaller, the width of the altered portion 13 can be made smaller than 0.5 μm. In addition, the width of the transformed portion 13 can be made larger than 5 μm as long as the film 12 is not removed by the laser beam 16.

The bonding state between atoms in the material of the film 12 contained in the transformed portion 13 is changed from the bonding state between atoms in the material contained in the film 12. For example, in the transformed portion 13, the crystalline material of the film 12 can be partially changed to be amorphous. As an example, if the film 12 is a diamond film, the transformed portion 13 may contain non-diamond carbon. Therefore, when a part of the film 12 is transformed into the transformed portion 13 during fabrication of the channel element 10, the volume of the transformed portion 13 expands more than the volume of the film 12 prior to the transformation. Thus, the mass density of the transformed portion 13 is lower than the mass density of the film 12.

As shown in FIG. 3, when the channel element 10 is fabricated, the laser light 16 is irradiated from an objective lens 15 to the channel element 10 having a film 12 formed on a substrate 11 before the channel is formed to form a transformed portion in line shape. Here, “in line shape” indicates that the position of the channel element 10 irradiated with the laser light 16 is relatively moved so as to draw a line. Moreover, the shape of the portion irradiated with the laser light 16 is not limited to straight lines and includes curved lines.

The objective lens 15 is positioned so as to irradiate the channel element 10 with the laser light 16 from a laser light source as converging light. The laser light 16 may be directed toward the film 12 as a convergent beam that converges near the interface between the substrate 11 and the film 12. The optical axis of the objective lens 15 may be oriented perpendicular to substrate 11. Also, the objective lens 15 may be adjusted in distance from the substrate 11 so that the laser light 16 is focused at or near the interface between the substrate 11 and the film 12. Further, as described above, the absorption rate of the laser light 16 of the film 12 can be higher on the side of the substrate 11. As a result, transformation of the film 12 occurs near the substrate 11.

Pulsed laser light emitted from a pulsed laser can be used as the laser light 16. The pulsed laser can be, for example, an ultrashort pulsed laser, such as a femtosecond laser. The ultrashort pulsed laser may be a pulsed laser with a pulse width in the range of 10⁻¹⁵ seconds to 10⁻¹² seconds. By using an ultrashort pulse laser, it is possible to irradiate the channel element 10 with the laser light 16 with pinpoint accuracy and without damaging a peripheral portion of the film 12. In particular, the photons produced by femtosecond lasers transfer their energy to a material at a short timescale relative to that of heat diffusion. Consequently, only material properties close to the spot where the photons are focused are transformed. It should be noted that the laser light 16 is not limited to a pulse laser and may be that from a continuous wave (CW) laser. The wavelength of the laser light 16 is a predetermined wavelength. The predetermined wavelength may refer to a particular range of wavelengths. When the film 12 is irradiated with a plurality of laser light 16 having different wavelengths, the predetermined wavelength may include a plurality of wavelengths. When using femtosecond lasers, the wavelength of laser light 16 is typically in the range of 1 μm to 1.1 μm.

At least part of the energy of the laser light 16 is absorbed by the film 12. As a result, the portion of the film 12 irradiated with the laser light 16 is transformed to be the transformed portion 13 extending in line shape along the substrate 11. Due to the expansion of the transformed portion 13, part of the film 12 is pushed away from the substrate 11 by the transformed portion 13. As a result, the substrate 11 and the film 12 are delaminated along the transformed portion 13 on both sides of the transformed portion 13. By this delamination, channels 14 surrounded by the substrate 11, the film 12 and the transformed portion 13 are formed on both sides of the transformed portion 13.

The dimension of the channels 14 in the direction perpendicular to the substrate 11 is preferably within the range of 5 nm to 200 nm, and more preferably within the range of 5 nm to 80 nm. This is because fluids confined in such channels 14 exhibit behaviors not observed in larger channels and because molecules, such as biomolecules with a size of several nanometers to several tens of nanometers, can be handled one by one in such channels. Channels smaller than such dimensions below may be difficult to fabricate due to, for example, fluctuations in the optical properties of the film 12, fluctuations in laser light 16 energy, fluctuations in the focal spot 16a position of the laser light 16. In addition, since the channel 14 is a delaminated portion between the substrate 11 and the film 12, the width of the channel 14 in the direction along the substrate 11 is several tens of times greater than the dimension in the direction perpendicular to the substrate 11. Typically, when the dimension in the direction perpendicular to the substrate 11 is 5 nm to 80 nm, the width of the channels 14 in the direction along the substrate 11 is preferably within the range of 1 μm to 4 μm. In addition, when the dimension in the direction perpendicular to the substrate 11 is 5 nm to 200 nm, the width of the channels 14 in the direction along the substrate 11 is preferably within the range of 1 μm to 10 μm.

It should be noted that more than two channels 14 can be formed in the channel element 10 by irradiating the channel element 10 with the laser light 16 so as to depict a plurality of lines. Further, by shifting the position irradiated with the laser light 16 at an appropriate pitch, the positions where the channels 14 are formed can be overlapped. Thereby, an odd number of channels 14 of 3 or more can be formed.

As described above, the channel element 10 can be easily formed by irradiating the film 12 formed on the substrate 11 with the laser light 16. Since the channels 14 of the channel element 10 formed by irradiation with the laser light 16 are surrounded by the substrate 11, the film 12, and the transformed portion 13, the process for sealing the channels that is required in the prior art is not necessary. Further, by using a femtosecond laser as the light source of the laser light 16 for forming the channels 14, it is possible to form fine channels with a width of 1 μm to 4 μm and a height of 5 nm to 80 nm. Furthermore, when glass is used as the material of the substrate 11 and poly-crystalline diamond is used as the material of the film 12, the channel element 10 is made of transparent materials, such that the channels 14 can be observed from the outside by optical means. Glass and diamond are advantageous in that they are thermally and chemically robust and biocompatible. Furthermore, glass and diamond are sustainable materials because they are made of elements that are abundant on the surface of the earth.

### Nanofluidic Device

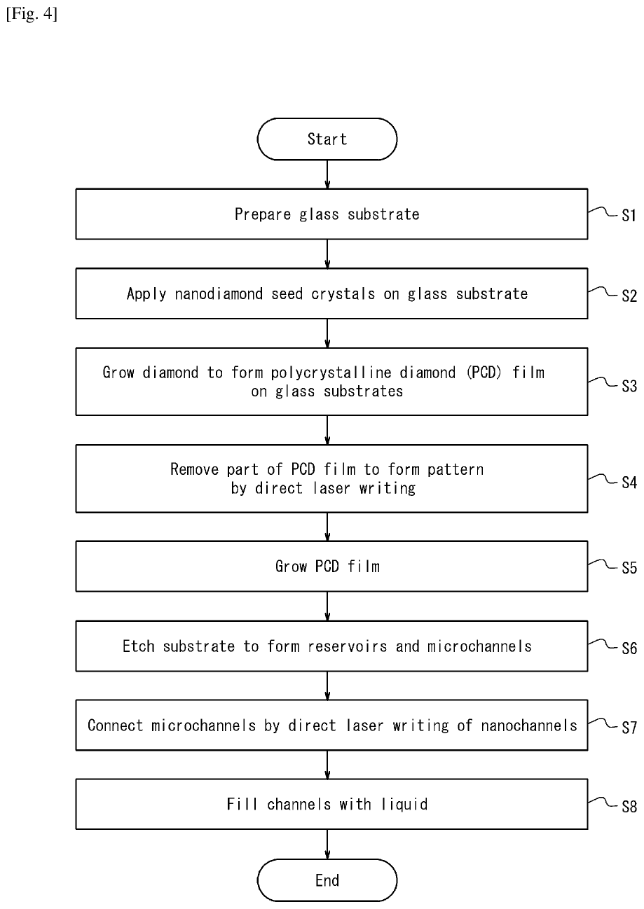

A “channel element” can also be referred to as a “channel device” or a “channel structure”. The channel element 10 may further include other components. Also, the channel element 10 may be incorporated into another device. A nanofluidic device 20 will be described below as an example of the channel element 10 of the present disclosure. First, a method for manufacturing the nanofluidic device 20 will be described with reference to the flow chart of FIG. 4 and the drawings corresponding to each step of the flow chart of FIG. 4.

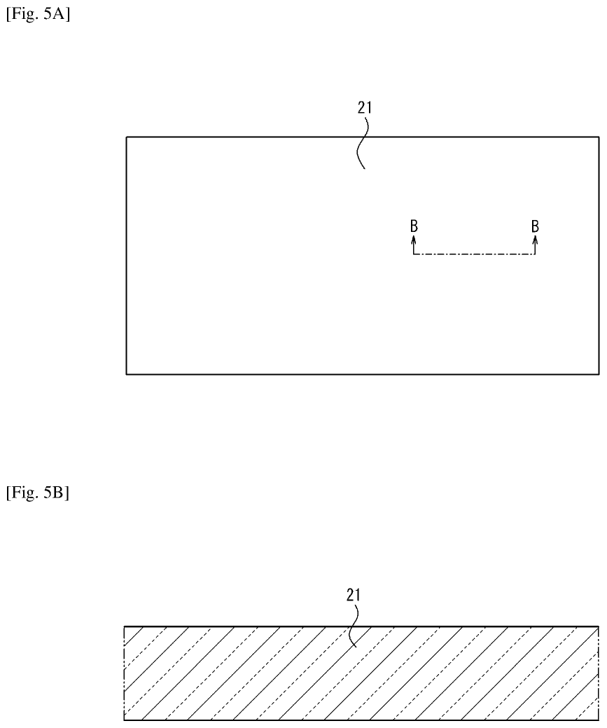

First, as shown in FIGS. 5A and 5B, a glass substrate 21 for manufacturing the nanofluidic device 20 is prepared (step S1). A flat, uniform glass substrate 21 can be used.

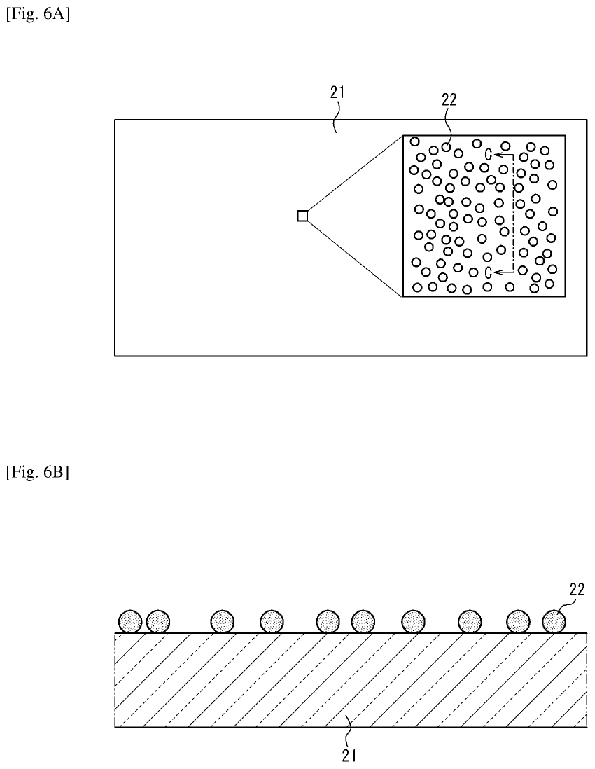

As shown in FIGS. 6A and 6B, nanodiamond seed crystals 22 are applied onto the glass substrate 21 (step S2). Nanodiamonds are nanoparticles that have the crystal structure of diamond. Nanodiamonds have the excellent properties of diamonds such as high transparency of light. FIGS. 6A and 6B show enlarged nanodiamond seed crystals 22 applied on the glass substrate 21.

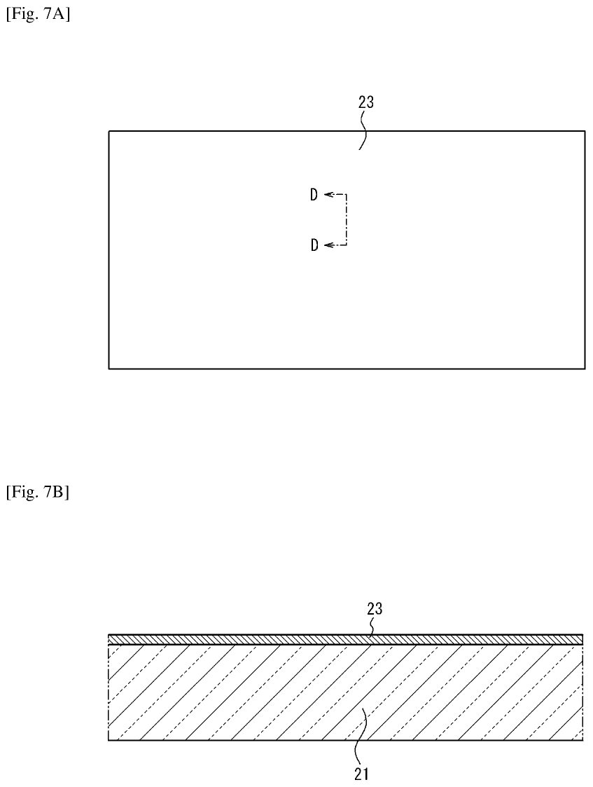

As shown in FIGS. 7A and 7B, diamond is grown on these nanodiamond seed crystals 22 by chemical vapor deposition until a polycrystalline diamond (PCD) film 23 of a first thickness (e.g., d=60 nm) is formed (step S3). The PCD film 23 formed on the glass substrate 21 has a uniform thickness. In one embodiment, polycrystalline diamond (PCD) film may be nanocrystalline diamond (NCD) film.

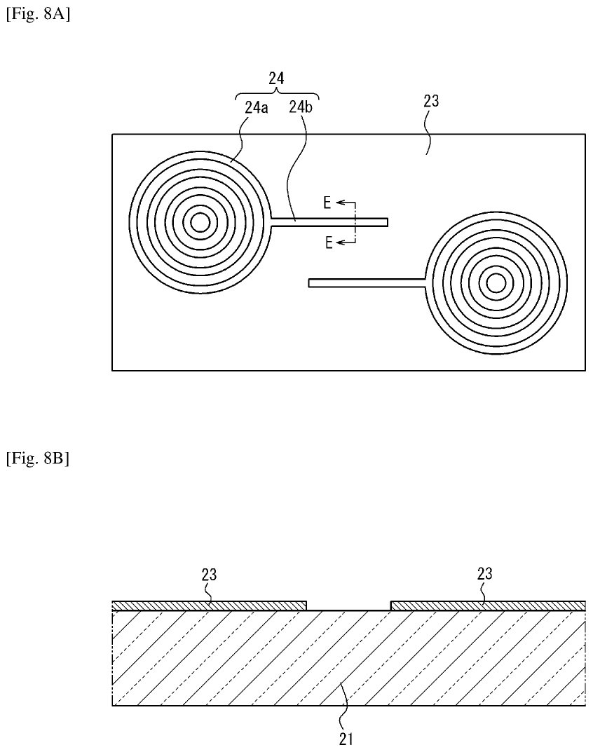

As shown in FIGS. 8A and 8B, a pattern 24 is formed on the PCD film 23 by removing portions of the PCD film 23 by direct laser writing (step S4). Since the PCD film 23 is kept relatively thin, patterning can be done at relatively low laser power to minimize damage of the glass substrate 21. The pattern 24 includes 2 pairs of concentric reservoir portions 24a and microchannel portions 24b extending linearly from the reservoir portion 24a. The two reservoir portions 24a are located apart from each other. The two microchannel portions 24b are arranged so as to face each other in parallel.

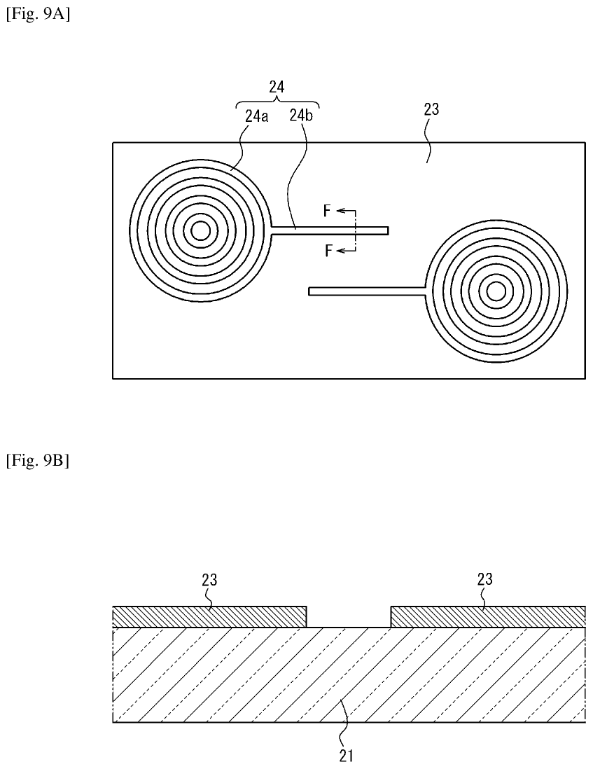

As shown in FIGS. 9A and 9B, the PCD film 23 is grown to a second thickness (e.g., d=300 nm) (step S5) by chemical vapor deposition. The second thickness should be sufficiently large to avoid PCD film 23 removal by laser irradiation. To obtain a dimension of the channels 14 in the direction perpendicular to the substrate 11 in the range 5 nm to 80 nm, a second thickness is preferably within the range of 280 nm to 320 nm. The second thickness is not limited to this range. The second thickness can be within the range of 145 nm to 1000 nm.

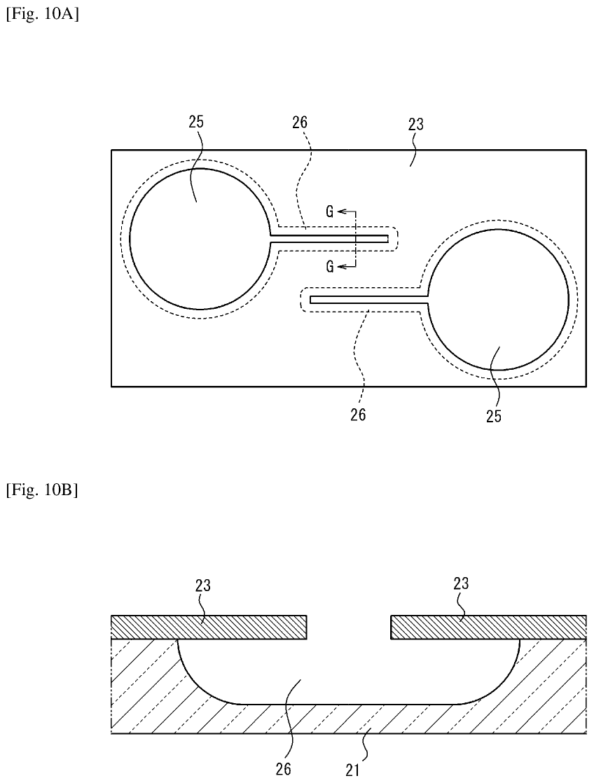

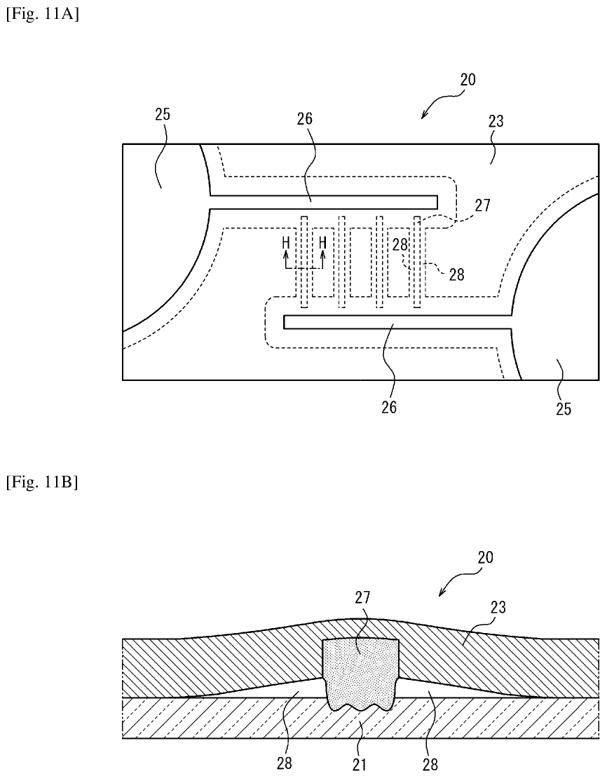

As shown in FIGS. 10A and 10B, the glass substrate 21 is etched with a hydrofluoric (HF) acid-based mixture. As a result, reservoirs 25 which can store fluid are formed at the positions of the reservoir portions 24a of the pattern 24, and microchannels 26, with an open top formed under the microchannel portions 24b (step S6) of the pattern 24. The microchannels 26 correspond to the second channels. Diamond does not react with hydrofluoric acid. Therefore, under the PCD film 23, the glass substrate 21 in a wider range than the pattern 24 formed on the PCD film 23 is removed. Each microchannel 26 is connected to a reservoir 25.

As shown in FIGS. 11A and 11B, microchannels 26 are connected through direct laser writing of nanochannels 28 (step S7). Nanostrips 27 (transformed portion) are formed mainly on the PCD film 23 side of the interface between the glass substrate 21 and the PCD film 23 by the laser light irradiation. Due to the expansion of the PCD film 23 when it transforms into the nanostrips 27, delamination occurs between the glass substrate 21 and the PCD film 23 to form nanochannels 28. As shown in FIGS. 11A and 11B, by using the channel forming method of the present disclosure, it is possible to form a large number of nanochannels parallel to each other.

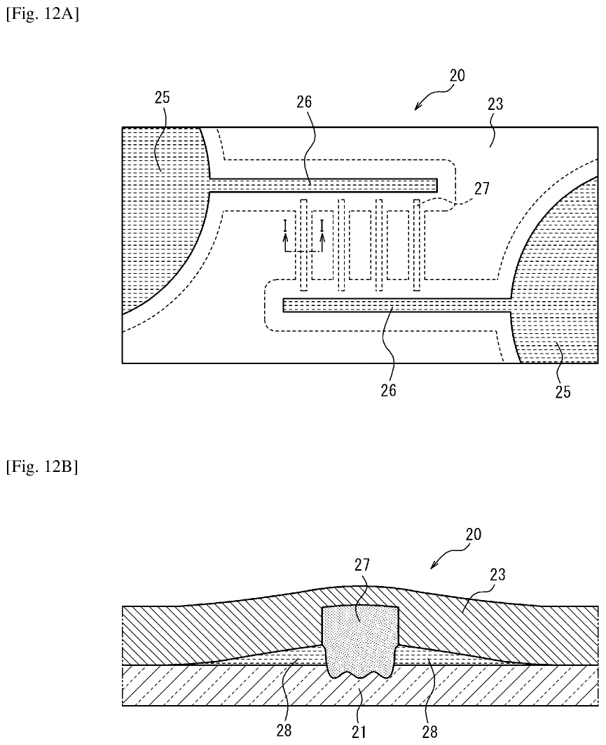

As shown in FIGS. 12A and 12B, the reservoirs 25 are filled with a liquid (step S8). The liquid is, for example, water. Capillary action fills the microchannels 26 and nanochannels 28 with the liquid. According to experiments conducted by the inventors of the present invention, it has been found that the liquid does not flow to all of the microchannels 26 and the nanochannels 28 simply by filling one reservoir 25 with liquid. In order to fill all of the microchannels 26 and the nanochannels 28 with liquid, both reservoirs 25 need to be filled with the liquid. In this way, a liquid to be tested or observed can be introduced into nanochannels 28.

In steps S4 and S7, a femtosecond laser can be used for direct laser writing. The inventors of the present invention used pulsed laser light with a laser pulse frequency of 756 kHz, a pulse width of 270 fs, a writing speed of 200 mm/s, and a wavelength of 1033 nm, and condensed the laser light to a spot with an approximate radius of 1 μm to fabricate the nanofluidic device 20.

The above explanation presents a method for the direct femtosecond laser writing of nanochannels 28 at the interface of the PCD film 23 and the glass substrate 21. The laser writing transforms a portion of the PCD film 23 into a nanostrip 27 which is the transformed portion 13. The nanostrip 27 is surrounded by two nanochannels 28 that are formed through delamination of the PCD film 23. For an PCD film 23 of d=300 nm, the mean height and width of a nanochannel 28 are of the order of 30 nm and 2 μm, respectively, and these dimensions can be tuned.

According to experiments conducted by the inventors of the present invention, the nanostrips 27 includes PCD, non-diamond carbon, and glass particles which might be mixed with each other by laser ablation. From this it follows that the expansion of the material shapes the nanostrips 27, and causes delamination and transformation of nanochannels 28. Such expansion occurs, for example, during transformation of diamond to non-diamond carbon.

Since ablation of a bare glass substrate only occurs at pulse energies significantly higher than those used for nanochannel writing, it is deduced that the formation of nanostrip 27 is initiated in the PCD film 23 near the interface of the PCD film 23 and the glass substrate 21. It is speculated that the origin of this phenomenon might be the presence of low-quality PCD at that location, which tends to absorb more light than high-quality PCD.

PCD is an attractive material for application in the nanofluidic device 20 due to its biocompatible, chemical, mechanical, electrical, optical, and quantum properties. Low-cost diamond films can be grown with microwave plasma-assisted chemical vapor deposition (MWPACVD). The PCD film 23 can be achieved on large-area substrates using methane gas diluted in molecular hydrogen as a precursor mixture after nanodiamond seeding. Adding oxygen into the mixture typically improves film quality, can lead to seed etching, and allows low temperature deposition. PCD can also be doped to make it electrically conductive, paving the way for various applications.

Being inexpensive, transparent, and chemically inert, glass is a natural choice for research in micro and nanofluidics. The properties of glass are also readily tunable. For example, glass can be made with a coefficient of thermal expansion (CTE) similar to the CTE of silicon, which is relatively close to the CTE of diamond. Glass can thus be used to fabricate structures with a relatively low residual stress. Since PCD is inert to hydrofluoric acid (HF), the PCD film 23 can act as an etch stop during the HF etching of glass. Here, the method of fabricating the nanofluidic device 20 relies on this property.

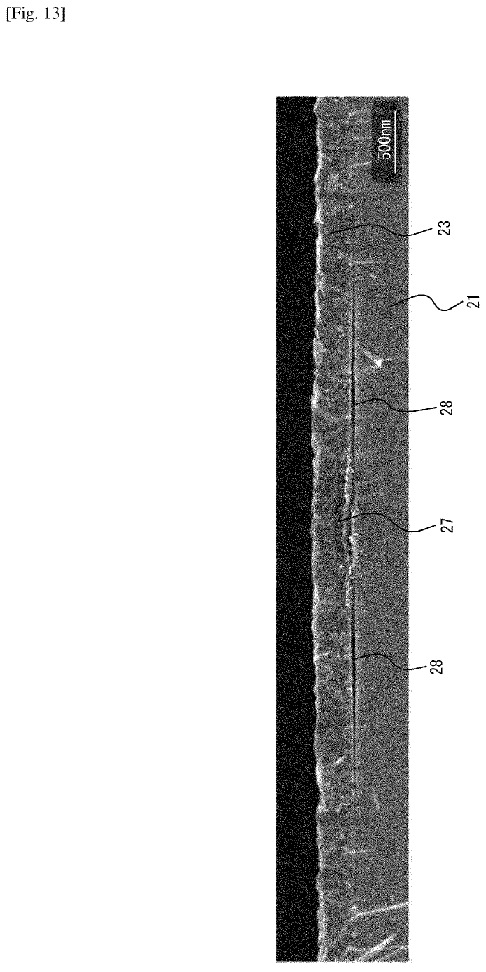

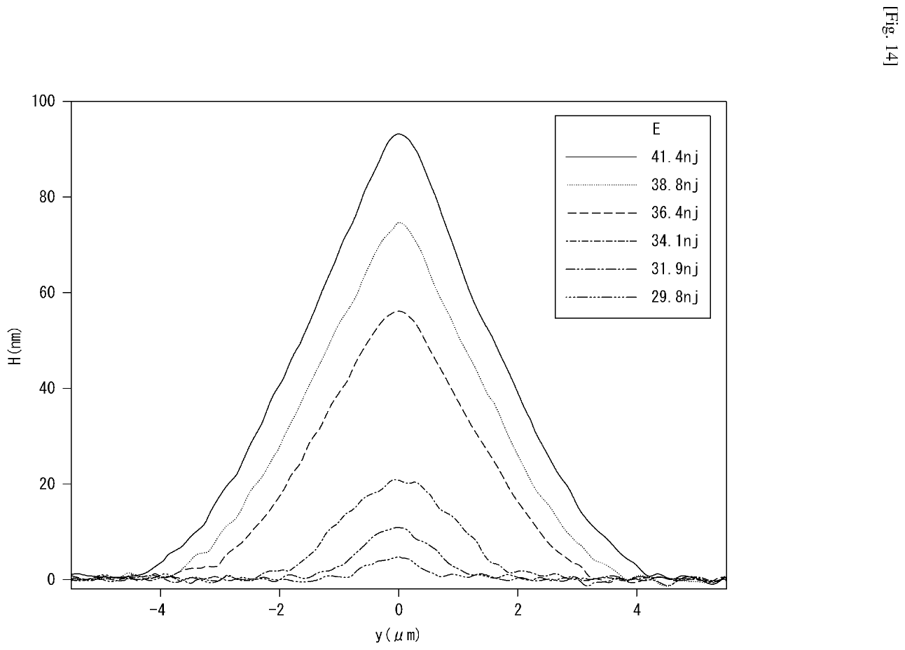

FIG. 13 depicts a scanning electron microscopy (SEM) image of a structure made by cleaving a sample of the nanofluidic device 20 right after film growth (d=300 nm), followed by laser writing over the cleaved edge with laser pulse energy E=36.4 nJ. The image shows the presence of a nanostrip 27 surrounded by two nanochannels 28 that are formed through delamination of the PCD film 23. Since laser ablation of a bare glass substrate occurs at laser pulse energy E>190 nJ, which is higher relative to the values of laser pulse energy E used for nanochannel writing, it is deduced that nanostrip formation is initiated in the PCD film 23. Since nanodiamond seed crystals 22 are highly defective and the diamond quality increases as the PCD film 23 grows, for the deposition conditions used in this process, the density of non-diamond material in an PCD film 23 increases towards the film-substrate interface. Such material is more likely to absorb laser light than diamond. Consequently, the portion of the PCD film 23 close to the glass substrate 21 tends to be transformed most by laser writing. When exposed to laser irradiation, the portion of the PCD film 23 close to the glass substrate 21 undergoes a sudden structural change, resulting in a decrease in mass density. The resulting nanostrip 27 has a lower mass density than the PCD film 23. Since the mass density of the irradiated portion decreases, the left and right peripheral portions are pushed and delaminated from the glass substrate 21 to form nanochannels 28. The PCD film 23 may be replaced with a film made of another material that has a lower laser light absorption rate in the upper portion and a higher laser light absorption rate in the lower portion. The another material is preferably chemically and thermally robust, biocompatible, and of which the optical properties can be tuned by controlling defect density.

FIG. 14 is a height profile of the air interface (that is, the upper surface of the PCD film 23) of each laser-written structure formed by varying the laser pulse energy E of the femtosecond laser in the manufacturing process of the nanofluidic device 20. In FIG. 14, the horizontal (abscissa) axis “y” is the distance from the center in the direction (y direction) orthogonal to both the direction (x direction) in which the nanostrip 27 extends and the direction (z direction) perpendicular to the glass substrate 21. The vertical (ordinate) axis “H” is the height of the air interface in the z direction. For the nanofluidic device 20, the height H of the top surface of the PCD film 23 before being irradiated with the femtosecond laser is set to zero. The height H is calculated by averaging the atomic force microscope (AFM) data over the x-direction.

As is clear from FIG. 14, the higher the laser pulse energy E, the greater the maximum value of the height profile of the nanofluidic device 20. That is, the higher the laser pulse energy E, the greater the deformation of the PCD film 23, so that larger nanochannels 28 are formed. When the laser pulse energy E is 36.4 nJ or more, the deformation of the PCD film 23 is relatively large, so relatively large nanochannels 28 are formed. On the other hand, when the laser pulse energy E is less than 29.8 nJ, the deformation of the PCD film 23 is small, so it may be difficult to form the nanochannels 28 satisfactorily.

The laser pulse energy E in Step S7 is selected from a range of energies in which the laser light 16 causes a certain degree of transformation in a part of the PCD film 23 but does not remove the PCD film 23. The appropriate magnitude of laser pulse energy E is determined based on various conditions including material of the PCD film 23.

The inventors of the present invention further prepared nanofluidic devices 20 by setting the thickness d of the PCD film 33 to various values in the range of 62 nm to 320 nm, and observed the nanochannels 28 with a reflected light microscope. As a result, it was confirmed that nanochannels 28 were formed in a relatively wide range of laser pulse energy E when the thickness d is 280 nm to 320 nm. Also, when the thickness d of the PCD film 33 was 300 nm, film removal due to laser irradiation was not observed for the range of appropriate laser pulse energy E. Since the deposition cost increases with increase of the thickness d, it is preferable to keep the thickness d relatively small. Another reason to keep the thickness d relatively small is that the surface roughness of PCD film 22 increases with increase of the thickness d. Surface roughness causes light scattering that hinders observing the channels with light mi-croscopes. Accordingly, the thickness of the PCD film 33 is preferably within the range of 280 nm to 320 nm. The thickness d of the PCD film 33 may be outside the preferred range of the thickness d described above. According to experiments conducted by the inventors of the present application, it was confirmed that the nanochannels 28 were formed when the thickness d was 145 nm or more. Further, it is presumed that the light scattering due to the surface roughness of the PCD film 33 is within the permissible range if the thickness of the PCD film 33 is up to about 1000 nm. Accordingly, the thickness of PCD film 33 is preferably within the range of 145 nm to 1000 nm. Thicker PCD films might require an additional polishing step to reduce scattering.

As described above, by using the channel forming method of the present disclosure, a nanofluidic device 20 having a large number of nanochannels 28 arranged in parallel can be manufactured. The nanofluidic device 20 is expected to be used for research and development, analysis, etc. in fields such as medicine, biotechnology, and chemistry. In addition, according to the channel formation method of the present disclosure, the size of the nanochannels 28 can be varied by changing the laser pulse energy E, etc. Therefore, the nanofluidic device 20 can be manufactured with nanochannels 28 having dimensions that correspond to various needs.

### Electronics Package

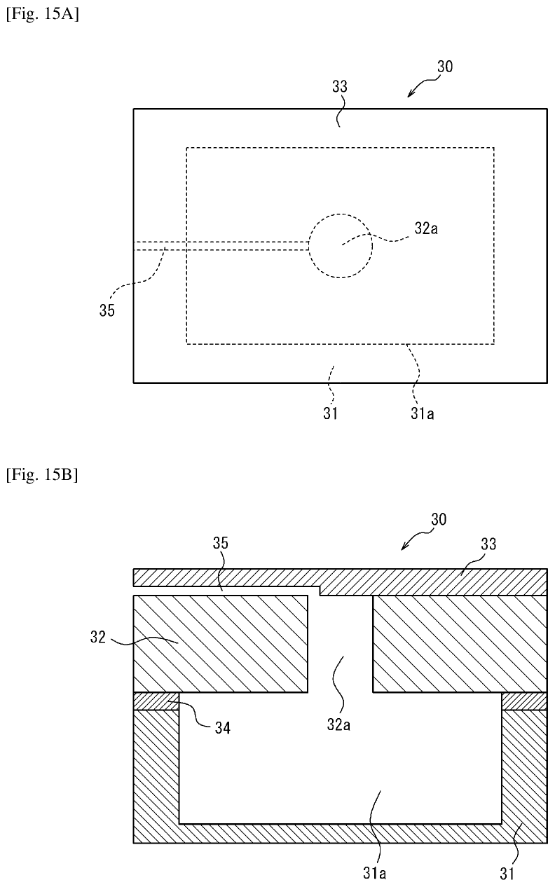

An electronics package 30 (herein after referred to as “the package 30”) including the channel element 10 of the present disclosure will be described with reference to FIGS. 15A and 15B.

The package 30 is formed by bonding a glass substrate 32 to the top of a package body 31 having a bottom and side portions with an adhesive 34. An electronic component can be placed in a hollow portion 31a, which is an internal space surrounded by the package 30 and the glass substrate 32. A via 32a is formed through the glass substrate 32 at the center of the glass substrate 32.

A PCD film 33 is formed on the glass substrate 32. The PCD film 33 covers the entire upper portion of the glass substrate 32 including the upper portion of the via 32a. Such an PCD film 33 can be formed, for example, by a known method described in WO2020/222297. Furthermore, between the glass substrate 32 and the PCD film 33, nanochannels 35 (first channels) are formed that connect the space inside the via 32a and the space outside the package 30. The nanochannels 35 are formed based on the method of manufacturing the channel element 10 shown in FIG. 3.

Since the package 30 is configured as described above, even if a solvent contained in the adhesive 34 evaporates and the pressure inside the hollow portion 31a increases, the gas inside the hollow portion 31a can be vented through the nanochannels 35. In addition, the nanochannels 35 can be hydrophobic and sufficiently small to block particles from the outer space. As a result, the environment around the electronic component inside the package 30 can be maintained in good condition.

### Drug Delivery Device

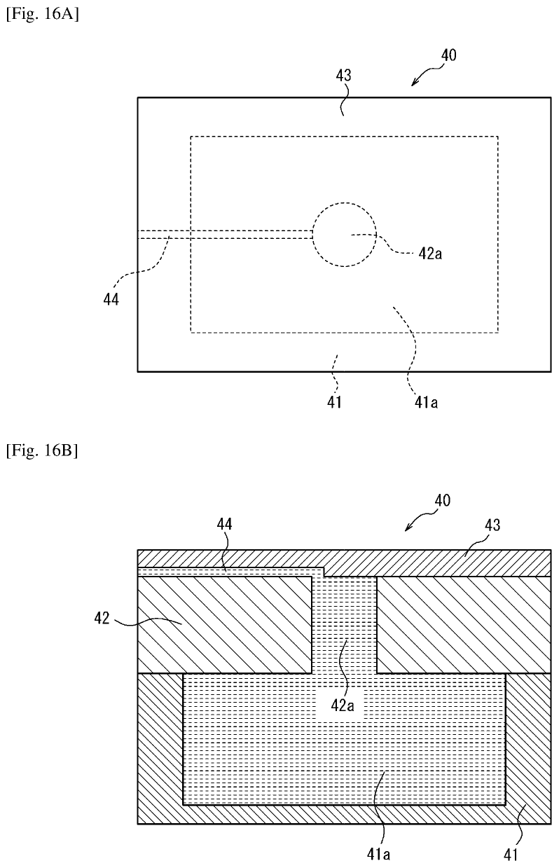

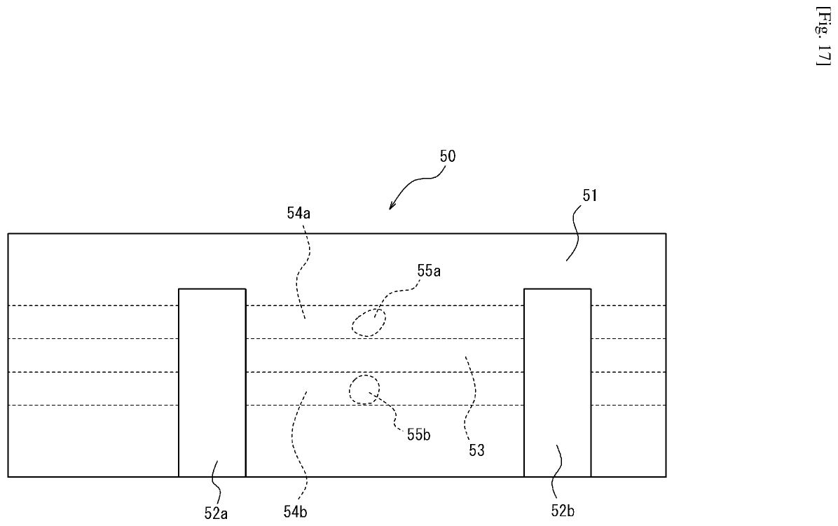

A drug delivery device 40 incorporating the channel element 10 of the present disclosure will now be described with reference to FIGS. 16A and 16B.

A drug delivery device 40 includes a housing portion 41 and a glass substrate 42. A hollow portion 41a surrounded by the housing portion 41 and the glass substrate 42 is filled with a drug during use. At the center of the glass substrate 42, a via 42a is formed through the glass substrate 42.

A PCD film 43 is formed on the glass substrate 42. The PCD film 43 covers the entire upper portion of the glass substrate 42 including the upper portions of the via 42a. Further, between the glass substrate 42 and the PCD film 43, drug delivery channels 44 (first channels) are formed to connect the space inside the drug delivery device 40 and the space outside the drug delivery device 40. The drug delivery channels 44 are channels used for drug delivery. The drug delivery channels 44 are formed based on the method of manufacturing the channel element 10 shown in FIG. 3.

The drug delivery device 40 can release the drug filled in the hollow portion 41a to a patient's desired site over a long period of time. Drug delivery device 40 uses drug delivery channels 44 as channels for releasing the drug. The dimensions of the drug delivery channels 44 can be set to an appropriate size according to the type of drug. The drug delivery device 40 may include a release mechanism (not shown) for releasing the drug inside the hollow portion 41a to the outside.

Since the drug delivery device 40 is configured as described above, it can release an appropriate drug to an appropriate site of a patient at an appropriate rate over a long period of time. Moreover, since the drug delivery device 40 is made of diamond and glass, it has an advantage in that it does not have a harmful effect on the human body.

### Biomolecule Testing Apparatus

A biomolecule testing apparatus 50 incorporating the channel element 10 of the present disclosure will now be described with reference to a plan view of FIG. 17.

The biomolecule testing apparatus 50 includes a channel element 51 according to the present disclosure, a first electrode 52a and a second electrode 52b. The channel element 51 comprises a nanostrip 53 and two nanochannels 54a and 54b. The channel element 51 uses a glass substrate as the substrate 11 of the channel element 10 and an PCD film as the film 12 in the same manner as the nanofluidic device 20 shown in FIGS. 4 to 13. The width and height dimensions of the nanochannels 54a and 54b can be tens to hundreds of nanometers. In addition, the substrate 11 and the film 12 of the biomolecule testing device 50 are not limited to the glass substrate and the PCD film. The substrate 11 and the film 12 can be formed of a variety of materials, as long as they meet certain requirements.

The first electrode 52a and the second electrode 52b may comprise a conductive metallic material such as platinum, gold, silver, or copper. The first electrode 52a and the second electrode 52b are disposed on the top and/or bottom surface of the channel element 51 separated from each other in the direction along the nanochannels 54a and 54b. The first electrode 52a and the second electrode 52b may be positioned at or adjacent to opposite ends of the nanochannels 54a and 54b. The first electrode 52a and the second electrode 52b can apply an electric field to nanochannels 54a and 54b. The first electrode 52a and the second electrode 52b may generate an electrophoretic force within the nanochannels 54a and 54b to cause migration of biomolecules 55a and 55b disposed within the nanochannels 54a and 54b.

The biomolecule testing apparatus 50 may further include a circuit for controlling the potentials of the first electrode 52a and the second electrode 52b, and an observation device for observing the biomolecules 55a and 55b to be inspected and which are trapped in the nanochannels 54a and 54b. The observation device is, for example, an optical microscope or an atomic force microscope. The biomolecule testing apparatus 50 may also include a light source and an optical system for irradiating the biomolecules 55a and 55b with light. The circuit, the observation device, the light source, and the like, may be devices external to the biomolecule testing apparatus 50 instead of being part of the biomolecule testing apparatus 50.

With the configuration described above, the biomolecule testing apparatus 50 can observe biomolecules one by one. For example, the biomolecule testing apparatus 50 can observe the behavior of the biomolecules 55a and 55b with respect to application of an electric field and/or irradiation of light. Also, for example, since the nanochannels 54a and 54b have dimensions smaller than those of biological cells, the biomolecular testing apparatus 50 can reproduce the chemical reactions of proteins contained in biological cells in the intracellular environment.

The number of nanochannels 54a and 54b of the biomolecule testing apparatus 50 is not limited to two. The biomolecule testing apparatus 50 may be configured to include a nanochannel array in which a large number of nanochannels are arranged in parallel.

The channel element 10 of the present disclosure is not limited to the nanofluidic device 20, the package 30, the drug delivery device 40, and the biomolecule testing apparatus 50 described above, and various applications are possible. The channel element 10 can be utilized for transport, mixing, reaction, observation, etc., of minute amounts of biomolecules and/or chemical substances. Therefore, it is expected to find use in many fields, including medicine, biotechnology, drug development, chemistry and material development.

### Method for Forming the Microchannel

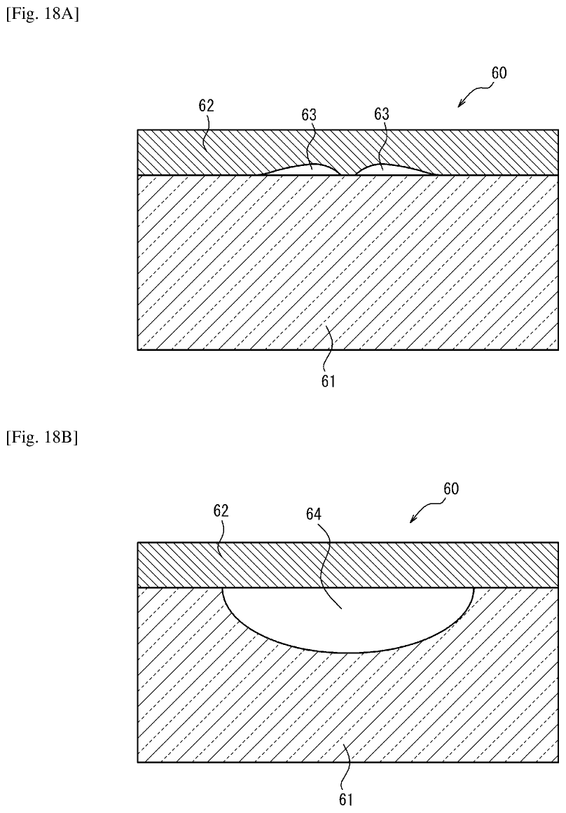

Referring to FIGS. 18A and 18B, a method of fabricating a channel element 60A containing a microchannel 64 having a width of 10 μm or more from a channel element 60 according to the present disclosure that contains two nanochannels 63 having a width of, for example, several hundreds nm to a few μm will be described. Nanochannels 63 correspond to the first channels and microchannel 64 corresponds to a third channel.

The channel element 60 includes a glass substrate 61 and an PCD film 62 formed on the glass substrate 61, similar to the nanofluidic device 20 shown in FIGS. 4 to 13.

Two nanochannels 63 are formed between the glass substrate 61 and the PCD film 62 according to the channel forming method of the present disclosure. FIG. 18A is a cross-sectional view along a plane perpendicular to the direction in which the two nanochannels 63 extend.

A liquid that corrodes the glass substrate 61 is filled in the nanochannels 63 of the channel element 60. The liquid that corrodes the glass substrate 61 is, for example, hydrofluoric acid (HF). By providing a reservoir at at least one end of the nanochannels and supplying hydrofluoric acid (HF) to this reservoir, the nanochannel 63 can be filled with hydrofluoric acid (HF) by capillary action.

As shown in FIG. 18B, by corroding the glass substrate 61 in contact with the nanochannels 63 with hydrofluoric acid (HF), the two nanochannels 63 are connected on the side of the glass substrate 61, forming a single microchannel 64 larger than the nanochannels 63 in width and height. Therefore, according to this channel forming method, it is possible to manufacture the channel element 60A in which the microchannel 64 is formed between the glass substrate 61 and the PCD film 62.

Although the embodiments of the present disclosure have been described with reference to drawings and examples, it should be noted that those skilled in the art can easily make various modifications or changes based on the present disclosure.

Therefore, it should be noted that these variations or modifications are included within the scope of this disclosure.

### REFERENCE SIGNS LIST

10 channel element
11 substrate
12 film
13 transformed portion
14 channels (first channels)
15 objective lens
16 laser light
16a focal spot
20 nanofluidic device
21 glass substrate (substrate)
22 nanodiamond seed crystal
23 polycrystalline diamond film (PCD film)
24 pattern
24a reservoir portion
24b microchannel portion
25 reservoir
26 microchannel(second channel)
27 nanostrip (transformed portion)
28 nanochannel (first channel)
30 package
31 package body
31a hollow portion
32 glass substrate (substrate)
32a via
33 polycrystalline diamond film (PCD film)
34 glue
35 nanochannel (first channel)
40 drug delivery device
41 housing portion
41a hollow portion
42 glass substrate
42a via
43 polycrystalline diamond film (PCD film)
44 drug delivery channels
50 biomolecule testing apparatus
51 channel element
52b first electrode
52b second electrode
53 nanostrip (transformed portion)
54a, 54b nanochannel
55a, 55b biomolecule

1. A channel element, comprising:
a substrate;
a film formed on the substrate; and
a transformed portion of a part of the film, the transformed portion extending in line shape along the substrate, wherein
first channels are formed along the transformed portion between the substrate and the film that is delaminated on both sides of the transformed portion.

2. The channel element according to claim 1, wherein
the transformed portion has a mass density lower than that of the film.

3. The channel element according to claim 1, wherein
the film includes a layer adjacent to the substrate having a higher absorption rate of light of a predetermined wavelength than the other part of the film.

4. The channel element according to claim 1, wherein
the substrate is a glass substrate.

5. The channel element according to claim 1, wherein
the film is a diamond film.

6. The channel element according to claim 5, wherein
the transformed portion includes non-diamond carbon.

7. The channel element according to claim 1, wherein
the film has a thickness in a range of 145 nm to 1000 nm.

8. The channel element according to claim 1, wherein
the transformed portion has a width in a range of 0.5 μm to 5 μm.

9. The channel element according to claim 1, wherein
a dimension of the first channel in a direction perpendicular to the substrate is in a range of 5 nm to 200 nm.

10. The channel element according to claim 1, wherein
width of the first channel in a direction along the substrate is in a range of 1 μm to 4 μm.

11. The channel element according to claim 1, further comprising
two second channels having a larger dimension than the first channels each connected to a reservoir capable of storing a fluid, wherein
the first channels connect between the two second channels.

12. A package comprising the channel element according to claim 1, wherein the first channels connect between an internal space of the package in which an electronic component can be arranged and a space outside the package.

13. A drug delivery device comprising the channel element according to claim 1, wherein the first channel is used as a channel for drug delivery.

14. A biomolecule testing apparatus comprising:
the channel element according to claim 1; and
a plurality of electrodes provided at a distance from each other along the first channels of the channel element.

15. A channel forming method including:
forming a film on a substrate; and
irradiating the film with a laser light to form a transformed portion in line shape in a part of the film, wherein the transformed portion causes delamination of the substrate and the film on both sides of the transformed portion to form first channels along the transformed portion between the substrate and the film.

16. The channel forming method according to claim 15, wherein
the portion of the film irradiated with the laser light expands to form the transformed portion and causes delamination of the substrate and the film on both sides of the transformed portion.

17. The channel forming method according to claim 15, wherein
the film includes a layer adjacent to the substrate having a higher absorption rate of the laser light than the other portions of the film.

18. (Canceled)

19. (Canceled)

20. The channel forming method according to claim 15, wherein
the laser light is applied to the film as a convergent beam that converges on an interface between the substrate and the film.

21. (canceled)

22. The channel forming method according to claim 15, wherein
energy of the laser light is selected from a range of energies in which the part of the film is transformed but the film is not removed by the laser light.

23. The channel forming method according to claim 15, further including
filling the first channels with a liquid that corrodes the substrate to enlarge the first channels on both sides of the transformed portion to form a single third channel.
